(19) United States
(12) Patent Application Publication
(10) Pub. No.: US 2025/0194979 A1
(43) Pub. Date: Jun. 19, 2025

(54) SYSTEMS AND METHODS FOR STRESS DETECTION

(71) Applicant: KONINKLIJKE PHILIPS N.V., EINDHOVEN (NL)

(72) Inventors: Eduard Gerard Marie Pelssers, EINDHOVEN (NL); Elise Claude Valentine Talgorn, EINDHOVEN (NL)

(21) Appl. No.: 18/849,828

(22) PCT Filed: Mar. 16, 2023
(86) PCT No.: PCT/EP2023/056722

§ 371 Date: Sep. 23, 2024

Related U.S. Application Data
(60) Provisional application No. 63/322,328, filed on Mar. 22, 2022.

(51) Int. Cl. A61B 5/16; G16H 20/10; G16H 50/70; G16H 80/00

(52) U.S. Cl. CPC A61B 5/165; G16H 20/10; G16H 50/70; G16H 80/00

(57) ABSTRACT

The present disclosure relates to systems and methods of detecting stress. As described herein, a system for measuring a specific detection of stress is provided that includes a plurality of physiological sensors coupled to a smart cable assembly that can provide a plurality of configurations of the physiological sensors. Physiological measurements are recorded and compared with stress profiles generated by a machine-learning algorithm to determine whether the patient is experiencing stress that should or can be treated by a healthcare specialist.

### FIELD OF THE DISCLOSURE

The present disclosure is directed generally to systems and methods for stress detection, and more specifically, to systems and methods of defining, identifying, and classifying of stress using physiological and contextual data associated with a patient.

### BACKGROUND

In the healthcare context, stress is typically defined as the non-specific response of the body to any demand made upon it. Because stress can have a variety of causes, it is often viewed as non-specific and is disregarded when treating patients in healthcare setting. However, evidence of stress can also be an indication that a patient's condition is worsening or about to decline, and/or can indicate a new or unaddressed medical condition. Nevertheless, valuable stress parameters are often overlooked and discarded by conventional diagnostics and clinical systems and methods.

### SUMMARY OF THE DISCLOSURE

The present disclosure relates to systems and methods for detecting and classifying stress. As described herein, measurements of different physiological parameters are recorded and compared with stress profiles generated by a machine-learning algorithm to determine whether the patient is experiencing stress that should or can be treated by a healthcare specialist. A healthcare specialist can then be alerted to the patient's condition and one or more appropriate actions can be taken.

According to an embodiment of the present disclosure, a stress detection system for detecting stress in a patient is provided. The system may comprise: at least a first smart cable assembly operatively connecting the patient to a patient monitor and configured to enable measurements from at least a first combination of physiological sensors. Each smart cable assembly may comprise a smart cable sub-assembly and the first combination of physiological sensors detachably coupled to the smart cable sub-assembly. The smart cable sub-assembly may include a medical cable operatively connected to an analog-to-digital converter and a universal connector. At least the first smart cable assembly may be further configured to enable measurements from at least a second combination of physiological sensors that is different from the first combination of physiological sensors, where at least the second combination of physiological sensors are also able to be detachably coupled to the smart cable sub-assembly.

In an aspect, the stress detection system may further comprise a patient monitoring system that includes: a port configured to receive the universal connector of at least the first smart cable assembly; and a controller. The controller may include at least one processor and a memory storing a stress detection component. The stress detection component may include instructions that, when executed by the at least one processor, perform one or more of the following: receive, from at least the first combination of physiological sensors, a first set of measured physiological parameters for the patient; compare the first set of measurement physiological parameters with a stress profile; determine, based on the comparison with the stress profile, whether the first set of physiological parameters indicate a detection of stress associated with a treatable medical condition; generate at least a first alert based on the determined detection of stress associated with the treatable medical condition; and transmit at least the first alert to a healthcare specialist associated with the patient.

In an aspect, the generated first alert may comprise a first recommended treatment for the patient. The first recommended treatment can include at least one of: a recommendation of administering an effective dose of a pharmaceutical composition to the patient; a recommendation of increasing a dosage of an existing pharmaceutical composition being administered to the patient; a recommendation of performing one or more medical procedures on the patient; and a recommendation of performing one or more additional medical tests on the patient.

In an aspect, the stress profile may comprise thresholds, durations, and weighting factors for a plurality of physiological parameters and is generated by a stress profile generator based on historical physiological information for a plurality of patients using a machine-learning algorithm.

In an aspect, the stress detection component may further include instructions that, when executed by the one or more processors, perform one or more of the following: receive, from the stress profile generator, the stress profile, wherein the stress profile generator comprises the machine-learning algorithm used to generate the stress profile; transmit, to the stress profile generator, at least the first set of measured physiological parameters; and receive, from the stress profile generator, an updated stress profile, wherein the updated stress profile comprises thresholds, durations, and weighting factors for a plurality of physiological parameters and is generated by the stress profile generator based on the historical physiological information for the plurality of patients and on at least the first set of measured physiological parameters.

In an aspect, the stress detection component may further include instructions that, when executed by the one or more processors, perform one or more of the following: reconfigure the stress detection system to enable at least the second combination of physiological sensors, wherein the second combination of physiological sensors is different from the first combination of physiological sensors; receive, from the second combination of physiological sensors, a second set of measured physiological parameters for the patient; compare the second set of measured physiological parameters with the stress profile; determine, based on the comparison with the stress profile, whether the second set of physiological parameters indicate a detection of stress associated with a treatable medical condition; generate at least a second alert based on the determined detection of stress associated with the treatable medical condition; and transmit at least the second alert to the healthcare specialist associated with the patient.

According to another embodiment of the present disclosure, a computer-implemented method for the detection of stress and the treatment of stress-related conditions is provided. The method may comprise: receiving, from a first combination of physiological sensors of a stress detection system, a first set of measured physiological parameters for a patient; comparing the first set of measured physiological parameters with a stress profile; determining, based on the comparison with the stress profile, whether the first set of measured physiological parameters indicate a detection of stress associated with a treatable medical condition; generating at least a first alert based on the determined detection of stress associated with the treatable medical condition; and transmitting at least the first alert to a healthcare specialist associated with the patient.

In an aspect, the stress detection system may comprise a first smart cable assembly including a smart cable sub-assembly and a plurality of physiological sensors operatively coupled to the smart cable sub-assembly, wherein the smart cable sub-assembly comprises a medical cable operatively connected to an analog-to-digital converter and a universal connector. The universal connector may be configured to operatively connect the first smart cable assembly to a patient monitor, and the first combination of physiological sensors may be a subset of the plurality of physiological sensors operatively coupled to the smart cable sub-assembly.

In an aspect, the stress profile may comprise thresholds, durations, and weighting factors for a plurality of physiological parameters and is generated based on historical physiological information for a plurality of patients using a machine-learning algorithm.

In an aspect, the method may further include: transmitting, to a stress profile generator, at least the first set of measured physiological parameters for the patient; receiving, from the stress profile generator, an updated stress profile, wherein the updated stress profile is generated based on the historical physiological information for the plurality of patients and at least the first set of measured physiological parameters for the patient using a machine-learning algorithm; receiving, from the first combination of physiological sensors of the first smart cable assembly, an updated set of measured physiological parameters for the patient; comparing the updated set of measured physiological parameters with the updated stress profile; determining, based on the comparison with the updated stress profile, whether the updated set of measured physiological parameters indicate a detection of stress associated with a treatable medical condition; generating an updated alert based on the determined detection of stress associated with the treatable medical condition; and transmitting the updated alert to a healthcare specialist associated with the patient.

In an aspect, the generated first alert may comprise a first recommended treatment for the patient. The first recommended treatment may comprise at least one of: a recommendation of administering an effective dose of a pharmaceutical composition to the patient; a recommendation of increasing a dosage of an existing pharmaceutical composition being administered to the patient; a recommendation of performing one or more medical procedures on the patient; and a recommendation of performing one or more additional medical tests on the patient.

In an aspect, the generated updated alert may comprise an updated recommended treatment for the patient. The updated recommended treatment may comprise at least one of: a recommendation of administering an effective dose of a pharmaceutical composition to the patient; a recommendation of increasing a dosage of an existing pharmaceutical composition being administered to the patient; a recommendation of performing one or more medical procedures on the patient; and a recommendation of performing one or more additional medical tests on the patient.

In an aspect, the method may further include determining, based on the comparison with the stress profile, an urgency level for the patient corresponding to a level of stress for the patient;

wherein at least the first generated alert further comprises the urgency level determined for the patient.

In an aspect, the method can further include: reconfiguring the stress detection system to enable a second combination of physiological sensors, wherein the second combination of physiological sensors is a subset of the plurality of physiological sensors operatively coupled to the smart cable sub-assembly, the second combination of physiological sensors being different from the first combination of physiological sensors; receiving, from the second combination of physiological sensors of the first smart cable assembly, a second set of measured physiological parameters for the patient; comparing the second set of measured physiological parameters with the stress profile; determining, based on the comparison with the stress profile, whether the second set of measured physiological parameters indicate a detection of stress associated with a treatable medical condition; generating at least a second alert based on the determined detection of stress associated with the treatable medical condition; and transmitting at least the second alert to a healthcare specialist associated with the patient.

In an aspect, the generated second alert comprises a second recommended treatment for the patient, the second recommended treatment comprising at least one of: a recommendation of administering an effective dose of a pharmaceutical composition to the patient; a recommendation of increasing a dosage of an existing pharmaceutical composition being administered to the patient; a recommendation of performing one or more medical procedures on the patient; and a recommendation of performing one or more additional medical tests on the patient.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1A is a schematic diagram of a stress detection system illustrated according to aspects of the present disclosure.

FIG. 1B is a schematic diagram of a stress detection system illustrated according to further aspects of the present disclosure.

FIG. 2 is a systematic diagram of a stress detection system including a stress detection component illustrated according to aspects of the present disclosure.

FIG. 3 is a flowchart illustrating a computer-implemented method for the detection of stress according to aspects of the present disclosure.

### DETAILED DESCRIPTION OF EMBODIMENTS

The present disclosure relates to systems and methods for detecting and classifying stress. While certain sources of stress may not be treatable, such as stress due to awareness of an upcoming surgery, other evidence of stress may be an indicator of a worsening of the patient's health that may, in fact, be treatable. As described herein, the systems and methods of the present disclosure leverage a specific detection of stress, which is often overlooked and discarded in conventional diagnostic systems and methods. The systems and methods of the present disclosure may be advantageously used to improve patient care and provide treatment to a patient.

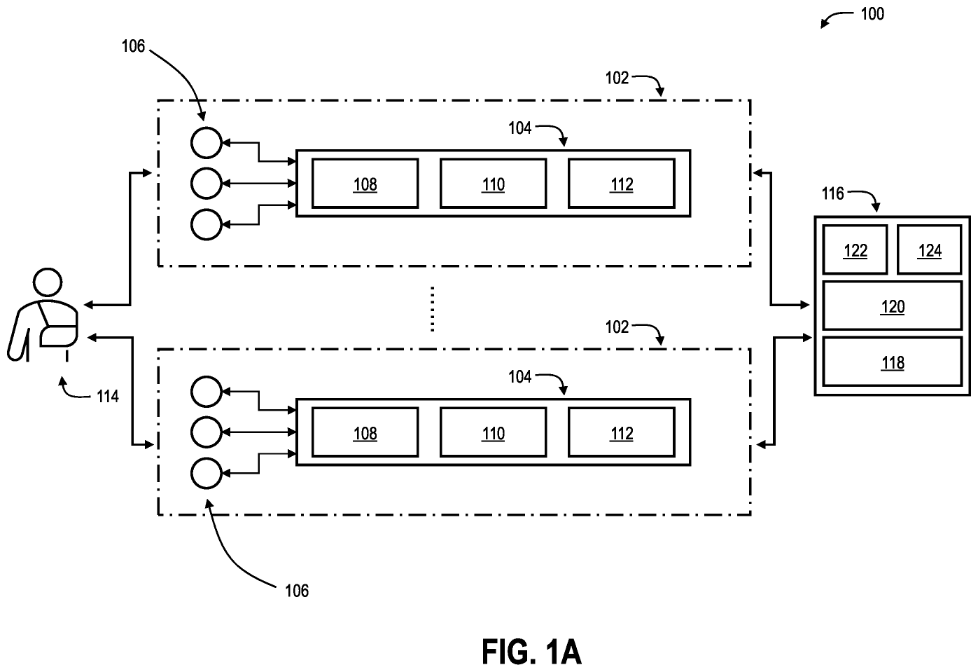

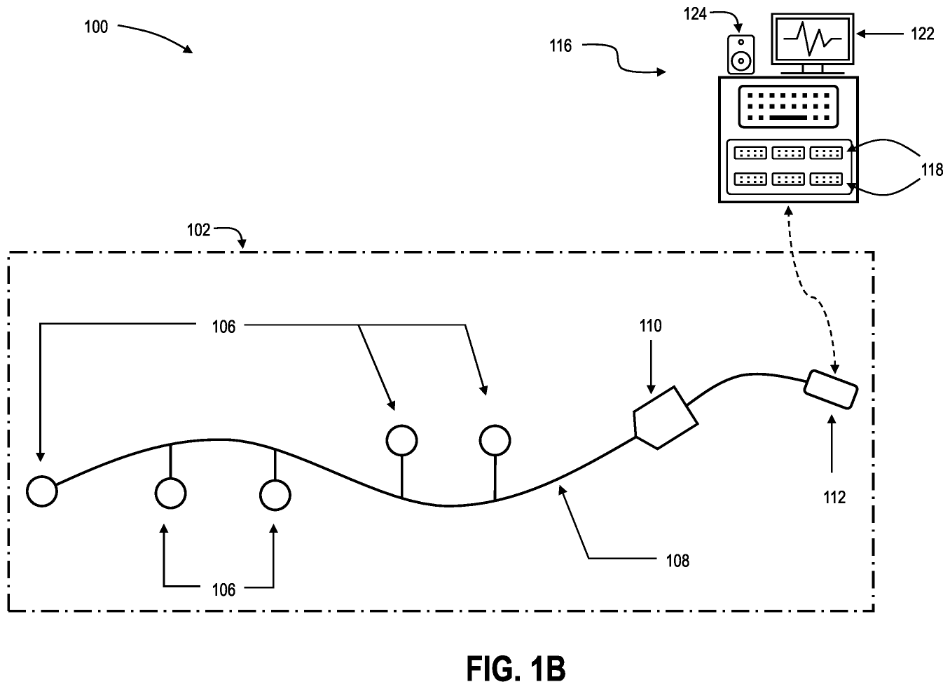

In accordance with an aspect of the present disclosure, a stress detection system 100 is provided. As shown in FIGS. 1A and 1B, the stress detection system 100 can include one or more smart cable assemblies 102 comprising a smart cable sub-assembly 104 and a plurality of physiological sensors 106 coupled to a corresponding smart cable sub-assembly 104. Each smart cable sub-assembly 104 can include, but is not limited to, a medical cable 108, an analog-to-digital converter 110, and a universal connector 112 that are operatively connected to the plurality of sensors 106 via the medical cable 108. In an aspect, the medical cable 108 can include multiple individual conductive wires, with each conductive wire being dedicated for one of the plurality of sensors 106. Alternatively, the medical cable 108 can include a single conductive wire and each sensor 106 of the plurality of sensors 106 may transmit signals in a manner that can be distinguished from the signals of the other sensors 106. In addition to converting an analog signal measured by the sensors 106 to a digital signal, the analog-to-digital converter 110 can include an electronic circuit that switches between the various sensors 106 and/or differentiates between the signals received from the combination of sensors 106.

As shown in FIG. 1A, the stress detection system 100 can include one or more such smart cable assemblies 102, where the sensors 106 of each smart cable assembly 102 is coupled to a patient 114 and a patient monitor 116. In specific embodiments, the stress detection system 100 includes at least a first smart cable assembly 102 with a first combination of sensors 106, where one or more of the sensors 106 are removably coupled to the smart cable sub-assembly 104. That is, one or more different types of sensors 106 can be removably attached to the smart cable sub-assembly 104 in a manner that allows for reuse of the smart cable sub-assembly 104. Thus, in an aspect, the sensors 106 can be disposable while the smart cable sub-assembly 104 can be sterilized, disinfected, or otherwise prepared for use with another patient 114.

In embodiments, the physiological sensors 106 can include different types of sensors operatively connected to the smart cable sub-assembly 104. For example, and without limitation, one or more of the physiological sensors 106 can be electrochemical sensors like conductive, amperometric, and/or potentiometric electrochemical-type sensors, or can be impedance sensors like a resistive and/or capacitive impedance-type sensor. In further embodiments, the physiological sensors 106 can include biokinetic sensors configured to record the movement of the patient and/or ambient sensors configured to measure environmental conditions of the patient (e.g., humidity, light, sound, temperature, etc.). In specific embodiments, the physiological sensors 106 can include one or more sensors 106 configured to measure physiological parameters such as heart rate, blood pressure, sweat/perspiration rate, respiration rate, oxygen saturation, cortisol levels, alertness, body temperature, one or more features of a patient's blood (e.g., glucose, pH, etc.), and the like.

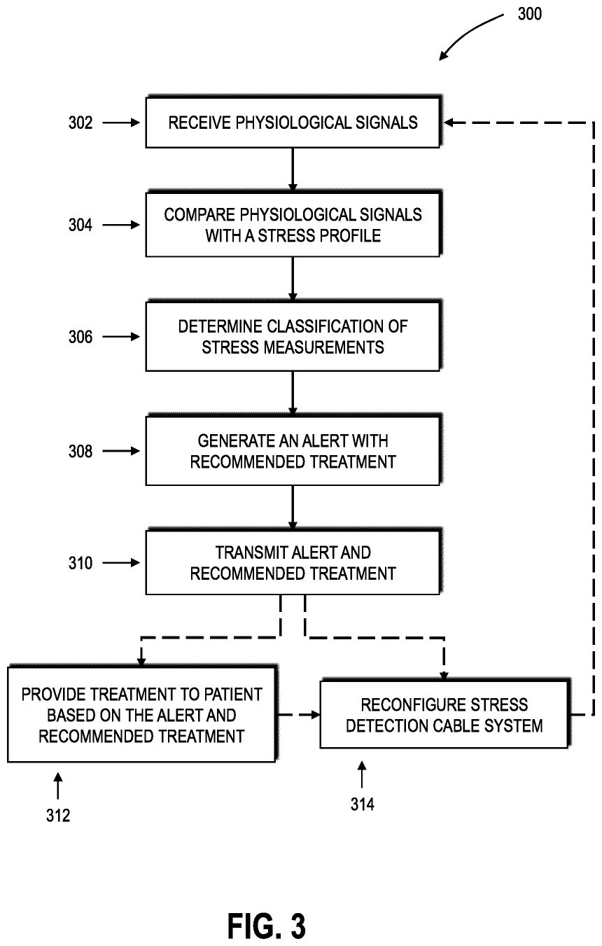

Additionally, the plurality of physiological sensors 106 can be reversibly coupled to the smart cable sub-assembly 104 in one or more different configurations. In a first configuration, the stress detection system 100 can enabled a first combination of sensors 106 to take measurements for a first set of physiological parameters. Then, in a second configuration, the stress detection system 100 can enable a second combination of sensors 106 to take measurements for a second set of physiological parameters, where the second set of sensors 106 and the second set of physiological parameters are wholly or partially different from the first set of sensors 106 and the first set of physiological parameters. As discussed below with respect to FIG. 3, the smart cable sub-assemblies 102 can be reconfigured either by enabling and/or disabling one or more connected sensors 106, or by physically interchanging one or more sensors 106 coupled to the sub-assembly 104.

Accordingly, the stress detection system 100 and the smart cable assemblies 102 enable the continuous, simultaneous, and/or semi-continuous measurement of one or more sets of physiological parameters, ranging from vital signs (e.g., heart rate, blood pressure, etc.), physiological responses (e.g., sweat rate, facial expression, body movements, etc.), and alertness (e.g., reaction to another person, coherence in speaking, introvertedness, etc.). In this manner, the stress detection systems 100 of the present disclosure can be configured in a plurality of different combinations of physiological sensors 106, thereby enabling the collection and customization of measured physiological signals.

With further reference to FIGS. 1A and 1B, the stress detection system 100 can include a patient monitoring system 116 connected to the one or more smart cable assemblies 102. In certain aspects, the patient monitoring system 116 can include one or more ports 118 configured to receive the universal connector 112 of one or more smart cable assemblies 102, and a controller 120. The patient monitoring system 116 can further include one or more additional components, such as a display 122 and/or audio equipment 124 (e.g., a speaker). In embodiments, the controller 120 can be configured to operate at least the stress detection system 100 and perform at least the methods describes herein. Additionally, the patient monitoring system 116 can be configured to use conventional devices to measure and/or display vital signs or other medical information useful in healthcare environment.

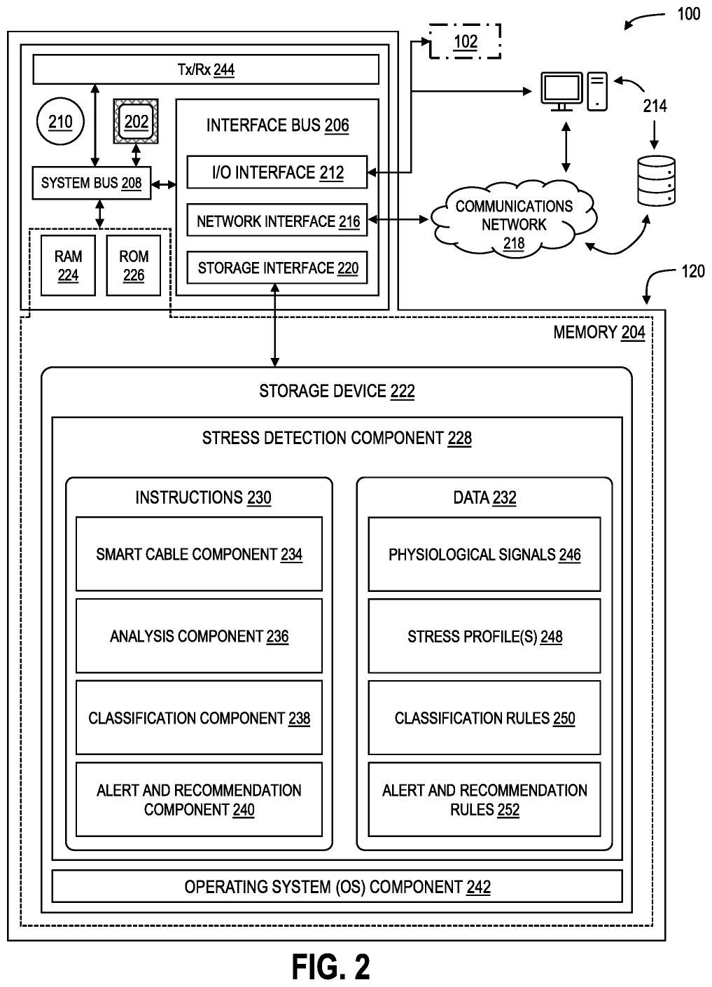

Turning to FIG. 2, these and other aspects of the controller 120 are described in further detail. As shown, the controller 120 can include at least one processor 202 (also referred to as central processing units or CPUs), machine-readable memory 204, and an interface bus 206, all of which may be interconnected and/or communicate through a system bus 208 containing conductive circuit pathways through which instructions (e.g., machine-readable signals) may travel to effectuate communications, tasks, storage, and the like. The controller 120 may be connected to a power source 210, which may include an internal power source and/or an external power source. The controller 120 can further include transceivers 244 that facilitate wireless communication, including wireless communication between the controller 120 and one or more of the peripheral devices 214.

In embodiments, the at least one processor 202 can comprise a high-speed data processor adequate to execute program components, which may include various specialized processing units as may be known in the art. The general processor may be a microprocessor, or may also be any traditional processor, controller, microcontroller, or state machine.

In embodiments, the interface bus 206 may include an input/output interface 212 configured to connect the patient monitoring system 116 to one or more smart cable assemblies 102 (e.g., via universal connectors 112) and/or to one or more peripheral devices 214. In particular aspects, the peripheral devices 214 can include one or more electronic devices, such as the following: graphics tablets; joysticks; keyboards; microphones; computer mouse (mice); touch screens (e.g., capacitive, resistive, etc.); trackballs; trackpads; styluses; audio devices; cameras; printers; video devices; and/or the like.

In an aspect, the interface bus 206 may include a network interface 216 configured to connect the patient monitoring system 116 to a communications network 218, which can include a direct interconnection, the Internet, a local area network (“LAN”), a metropolitan area network (“MAN”), a wide area network (“WAN”), a wired or Ethernet connection, a wireless connection, and similar types of communications networks, including combinations thereof. In this manner, the controller 120 can be connected to or in communication with one or more peripheral devices 214 via the communications network 218.

In an aspect, the interface bus 206 may further include a storage interface 220 configured to accept, communicate, and/or connect to a number of machine-readable memory devices, such as storage device 222, a removable storage device, or the like.

In embodiments, the memory 204 can be variously embodied in one or more forms of machine-accessible and machine-readable memory, including a various types of storage devices 222, random access memory 224, and read-only memory 226. In aspects, the storage device 222 can include a non-transitory storage medium, a magnetic disk storage, an optical disk storage, an array of storage devices, a solid-state memory device, and the like, including combinations thereof.

In embodiments, the memory 204 can include a stress detection component 228 that includes a collection of program (and/or database) components 230 and/or data 232. Depending on the particular implementation, the stress detection component 228 may include software components, hardware components, and/or some combination of both hardware and software components.

In particular embodiments, the memory 204 can include an operating system (“OS”) component 242, while the stress detection component 228 can include, but is not limited to, instructions 230 having a smart cable component 234, an analysis component 236, a classification component 238, and an alert and recommendation component 240. One or more of these components may be stored and accessed from the storage device 222 accessible through the interface bus 206, or can be loaded and/or stored in memory 204 via certain peripheral devices 214 (e.g., external memory devices, remote storage devices, and the like). That is, the aforementioned components may be incorporated into, loaded from, loaded onto, or otherwise operatively available to and from the stress detection component 228. Similarly, the stress detection component 228 can be incorporated into, loaded from, loaded onto, or otherwise operatively available to and from the controller 120 of the patient monitoring system 120. For example, although program components may be stored in a local storage device 222, they may also be loaded and/or stored in other memory, such as a remote cloud storage facility accessible through a communications network (e.g., communications network 218).

The operating system component 242 can be an executable program component facilitating the operation of at least the stress detection component 242. Typically, the operating system component 242 can facilitate access of the I/O, network, and storage interfaces, and can communicate with other components of the system 100.

The smart cable component 234 can be a stored program component that, when executed by at least one processor 202, may receive as an input the different physiological signals 246 measured using one or more connected smart cable assemblies 102, store these physiological signals 246 in the storage device 222, and reconfigure the stress detection system 100 to enable one or more combinations of physiological sensors 106 (including at least a second combination of physiological sensors 106 that is different from a prior combination of physiological sensors 106).

The analysis component 236 can be a stored program component that, when executed by at least one processor 202, may retrieve stress profiles 248 (discussed in more detail below) and analyze the physiological data 246 based on the retrieved stress profiles 246.

The classification component 238 can be a stored program component that, when executed by at least one processor 202, may determine whether a patient 114 falls into at least a first classification of stress management or a second classification of stress management based on certain predetermined classification rules 250.

The alert and recommendation component 240 can be a stored program component that, when executed by at least one processor 202, may generate an alert or recommendation based certain predetermined alert and recommendation rules 252, as well as transmit the alert or recommendation to a healthcare specialist associated with the patient 114 via a component of the patient monitor 116. In embodiments, the generated alerts can comprise a recommended treatment for the patient 114 for which the classification is performed.

Turning now to FIG. 3, a computer-implemented method 300 for the detection of stress is illustrated in accordance with these and further aspects of the present disclosure. In embodiments, the method 300 can include: at a step 302, receiving, from a first combination of physiological sensors 106 of a stress detection system 100, a first set of measured physiological parameters 246 for a patient 114; at a step 304, comparing the first set of measured physiological parameters 246 with a stress profile 248; at a step 306, determining, based on the comparison with the stress profile 248, whether the first set of measured physiological parameters 246 indicate a detection of stress associated with a treatable medical condition; at a step 308, generating at least a first alert based on the determined detection of stress associated with the treatable medical condition; and, at a step 310, transmitting at least the first alert to a healthcare specialist associated with the patient 114.

At the step 302, the method 300 can include receiving a first set of measured physiological parameters 246 associated with the patient 114 from a first combination of physiological sensors 106 of a stress detection system 100. As used herein, the term “physiological parameters” refers to any parameter associated with the physiology of a patient that may be measured. For example, a set of measured physiological parameters 246 can comprise physiological signals 246 measured using one or more physiological sensors 106.

At step 304, the method 300 can include comparing at least one set of physiological parameters 246 measured according to the present disclosure with a stress profile 248. As used herein, a stress profile 248 is a unique profiling correlation of multiple physiological parameters 26 and contextual patient data (e.g., outcomes, diagnoses, etc.). The stress profiles 248 can be developed based on at least historical physiological information for a plurality of patients. In embodiments, the stress profile 248 can comprise thresholds, durations, and weighting factors for a plurality of input parameters (e.g., physiological parameters). In aspects, the stress profile 248 may establish individual threshold values and/or defined ranges for parameters such as heart rate, blood pressure, alertness, respiration rate, and the like, or may establish multiple interdependent/conditional thresholds for such parameters (e.g., if heart rate is greater than a first threshold X, then the threshold for blood pressure Y1, but if the heart rate is less than threshold X, then the threshold for blood pressure is Y2, etc.). In further aspects, the stress profile 248 may establish expected or standard durations for different parameters as well as standard deviations for the parameters indicating a degree of severeness. For example, exceeding a threshold for a first, short period of time may not be indicative of a treatable stress-related condition, but exceeding the threshold repeatedly or for an extended period of time can be clinically relevant. In still further aspects, the stress profile 248 may establish weighting factors for different parameters or combinations of parameters. For example, different parameters may be given greater or lesser importance depending on the contextual patient information or may account for sudden and/or gradual changes in the patient's condition.

In embodiments, one or more stress profiles 248 may be generated by and received from a stress profile generator. In aspects, the stress profile generator can include a device executing machine-learning algorithm that receives at least historical physiological information for a plurality of patients and other contextual patient information, such as patient diagnoses, patient prognoses, and/or patient trajectories for a plurality of treatable and untreatable medical conditions. As such, given a set of physiological parameters 246 for a particular patient 114, the stress profile 248 can provide a specific detection of stress. That is, by comparing one or more sets of measured physiological parameters 246 with the stress profile 248, the patient's 114 indication of stress may be stratified and differentiated into one or more classes suggestive of a patient prognosis and/or a trajectory of the patient's 114 condition.

Next, in step 306, the method 300 can include determining, based on the comparison performed in step 304, whether the patient 114 falls into one or more classes of patients using a predefined set of classification rules 250. In embodiments, one classification of stress measurements may indicate a specific detection of stress due to non-medical sources and/or an untreatable condition. In further embodiments, another classification of stress measurements may indicate a specific detection of stress due to a treatable medical condition. In aspects, the non-medical/untreatable stress source can be, for example and without limitation, knowledge or aware of an upcoming surgery, issues with relationships or family, financial issues, and the like, which are not treatable by healthcare specialists. In further aspects, the treatable medical condition can be, for example and without limitation, inflammation, pain due to inflammation, pain due to physical trauma, a cardiovascular disorder, dehydration, a nutritional deficiency, and anxiety or restlessness due to a mental disorder. Thus, the first classification can indicate stress that may be handled by the patient without treatment or intervention by a healthcare specialist, while the second classification can indicate stress that will induce further worsening of a condition of the patient's 114 health and should be addressed/treated by the healthcare specialist.

In specific embodiments, step 306 includes determining whether one or more sets of measured physiological parameters 246 indicate a detection of stress associated with a treatable medical condition based on one or more comparisons with one or more stress profiles 248. In still embodiments, step 306 can include determining, based on the comparisons made in step 304, an urgency level for the patient corresponding to a level of stress of the patient 114. For example, the urgency level may indicate a period of time in which treatment should be provided.

Then, in step 308, the method 300 can include generating one or more alerts based on the determination made in step 306, such as the determined detection of stress associated with the treatable medical condition. In embodiments, the generated alert can include a recommended treatment for the patient based on one or more alert/recommendation rules 252. In certain aspects, the alert can also include the determined urgency level for the patient 114, and the recommended treatment can include a period of time in which treatment should be provided.

In step 310, the method 300 can include transmitting one or more generated alerts to a healthcare specialist associated with the patient 114 via a component of the patient monitor 116. For example, a component of the patient monitor 116 can include a display screen 122 or a speaker 124, and the alert can be a visual alert and/or an audible alert. In embodiments, the component of the patient monitor 116 can include a transmitter or other wireless communications device (not shown) that facilitates digital communication of the alert (e.g., a message to a pager or mobile device).

In response to receiving the alert in step 310, the method 300 can include the step 312, where a treatment is provided to the patient 114 based on the generated first alert and the recommended treatment. In embodiments, the treatment provided to the patient 114 can include, for example and without limitation, administering an effective dose of a pharmaceutical composition to the patient 114, increasing the dosage of an existing pharmaceutical composition being administered to the patient 114, performing one or more medical procedures on the patient 114, and/or performing one or more additional medical tests on the patient 114, and the like.

In particular embodiments, the method 300 can further include transmitting at least one set of measured physiological parameters and/or contextual patient information to the stress profile generator so that the stress profile generator can update the stored patient information used to generate subsequent stress profiles 248. As such, in embodiments, the method 300 can include receiving, from the stress profile generator, one or more updated stress profiles 248 that were generated based on the historical patient information as well as one or more sets of measured physiological parameters 246 from a current patient 114. Steps 306 through 314 of the method 300 can then be performed again or for the first time based on the updated stress profile 248. More specifically, the method 300 can then include the following steps: receiving, from the first combination of physiological sensors 106 of the stress detection system 100, an updated set of measured physiological parameters 246 for the patient 114; comparing the updated set of measured physiological parameters 246 with the updated stress profile 248; determining, based on the comparison with the updated stress profile 248, whether the updated set of measured physiological parameters 246 indicate a detection of stress associated with a treatable medical condition; generating an updated alert based on the determined detection of stress associated with the treatable medical condition; and transmitting the updated alert to a healthcare specialist associated with the patient 114. In embodiments, the generated updated alert can include an updated recommended treatment for the patient 114, and the updated recommended treatment can include at least one of: a recommendation of administering an effective dose of a pharmaceutical composition to the patient 114; a recommendation of increasing a dosage of an existing pharmaceutical composition being administered to the patient 114; a recommendation of performing a medical procedure n the patient 114; and a recommendation of performing one or more additional medical tests on the patient 114.

In still further embodiments, at a step 314, the method 300 can include reconfiguring the stress detection system 100 to enable at least a second combination of physiological sensors 106, wherein the second combination of physiological sensors 106 is different from the first combination of physiological sensors 106. As used herein, one combination of physiological sensors 106 is different from another combination of physiological sensors 106 if they enable one or more sensors 106 of the plurality of physiological sensors 106 coupled to the smart cable sub-assembly 104 in order to measure one or more physiological signals that were not measured by the previous combination of physiological sensors 106. As such, the steps 302 through 312 may be repeated for the second combination of physiological sensors 106 in order to generate at least a second alert and provide at least a second recommended treatment to the patient 114.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A stress detection system for detecting stress in a patient, the system comprising:
at least a first smart cable assembly operatively connecting the patient to a patient monitor and configured to enable measurements from at least a first combination of physiological sensors, wherein each smart cable assembly comprises a smart cable sub-assembly and the first combination of physiological sensors detachably coupled to the smart cable sub-assembly, the smart cable sub-assembly including a medical cable operatively connected to an analog-to-digital converter and a universal connector;
wherein at least the first smart cable assembly is further configured to enable measurements from at least a second combination of physiological sensors, the second combination of physiological sensors being different from the first combination of physiological sensors and also able to be detachably coupled to the smart cable sub-assembly.

2. The stress detection system of claim 1, further comprising:
the patient monitor, wherein the patient monitor comprises:
a port configured to receive the universal connector of at least the first smart cable assembly; and
a controller having at least one processor and a memory storing a stress detection component, wherein the stress detection component includes instructions that, when executed by the at least one processor, perform one or more of the following:
receive, from at least the first combination of physiological sensors, a first set of measured physiological parameters for the patient;
compare the first set of measurement physiological parameters with a stress profile;
determine, based on the comparison with the stress profile, whether the first set of physiological parameters indicate a detection of stress associated with a treatable medical condition;
generate at least a first alert based on the determined detection of stress associated with the treatable medical condition; and
transmit at least the first alert to a healthcare specialist associated with the patient.

3. The stress detection system of claim 2, wherein the generated first alert comprises a first recommended treatment for the patient, the first recommended treatment comprising at least one of: a recommendation of administering an effective dose of a pharmaceutical composition to the patient; a recommendation of increasing a dosage of an existing pharmaceutical composition being administered to the patient; a recommendation of performing one or more medical procedures on the patient; and a recommendation of performing one or more additional medical tests on the patient.

4. The stress detection system of claim 2, wherein the stress profile comprises thresholds, durations, and weighting factors for a plurality of physiological parameters and is generated by a stress profile generator based on historical physiological information for a plurality of patients using a machine-learning algorithm.

5. The stress detection system of claim 4, wherein the stress detection component further includes instructions that, when executed by the at least one processor, perform one or more of the following:
receive, from the stress profile generator, the stress profile, wherein the stress profile generator comprises the machine-learning algorithm used to generate the stress profile;
transmit, to the stress profile generator, at least the first set of measured physiological parameters; and
receive, from the stress profile generator, an updated stress profile, wherein the updated stress profile comprises thresholds, durations, and weighting factors for a plurality of physiological parameters and is generated by the stress profile generator based on the historical physiological information for the plurality of patients and on at least the first set of measured physiological parameters.

6. The stress detection system of claim 2, wherein the stress detection component further includes instructions that, when executed by the at least one processor, perform one or more of the following:
reconfigure the stress detection system to enable at least the second combination of physiological sensors, wherein the second combination of physiological sensors is different from the first combination of physiological sensors;
receive, from the second combination of physiological sensors, a second set of measured physiological parameters for the patient;
compare the second set of measured physiological parameters with the stress profile;
determine, based on the comparison with the stress profile, whether the second set of physiological parameters indicate a detection of stress associated with a treatable medical condition;
generate at least a second alert based on the determined detection of stress associated with the treatable medical condition; and
transmit at least the second alert to the healthcare specialist associated with the patient.

7. A computer-implemented method for the detection of stress, the method comprising:
receiving, from a first combination of physiological sensors of a stress detection system, a first set of measured physiological parameters for a patient;
comparing the first set of measured physiological parameters with a stress profile;
determining, based on the comparison with the stress profile, whether the first set of measured physiological parameters indicate a detection of stress associated with a treatable medical condition;
generating at least a first alert based on the determined detection of stress associated with the treatable medical condition; and
transmitting at least the first alert to a healthcare specialist associated with the patient.

8. The computer-implemented method of claim 7, wherein the stress detection system comprises a first smart cable assembly including a smart cable sub-assembly and a plurality of physiological sensors operatively coupled to the smart cable sub-assembly;
wherein the smart cable sub-assembly comprises a medical cable operatively connected to an analog-to-digital converter and a universal connector;
wherein the universal connector is configured to operatively connect the first smart cable assembly to a patient monitor; and
wherein the first combination of physiological sensors is a subset of the plurality of physiological sensors operatively coupled to the smart cable sub-assembly.

9. The computer-implemented method of claim 7, wherein the stress profile comprises thresholds, durations, and weighting factors for a plurality of physiological parameters and is generated based on historical physiological information for a plurality of patients using a machine-learning algorithm.

10. The computer-implemented method of claim 9, further comprising:
transmitting, to a stress profile generator, at least the first set of measured physiological parameters for the patient;
receiving, from the stress profile generator, an updated stress profile, wherein the updated stress profile is generated based on the historical physiological information for the plurality of patients and at least the first set of measured physiological parameters for the patient using a machine-learning algorithm;
receiving, from the first combination of physiological sensors of the first smart cable assembly, an updated set of measured physiological parameters for the patient;
comparing the updated set of measured physiological parameters with the updated stress profile;
determining, based on the comparison with the updated stress profile, whether the updated set of measured physiological parameters indicate a detection of stress associated with a treatable medical condition;
generating an updated alert based on the determined detection of stress associated with the treatable medical condition; and
transmitting the updated alert to a healthcare specialist associated with the patient.

11. The computer-implemented method of claim 7, wherein the generated first alert comprises a first recommended treatment for the patient and the first recommended treatment comprises at least one of: a recommendation of administering an effective dose of a pharmaceutical composition to the patient; a recommendation of increasing a dosage of an existing pharmaceutical composition being administered to the patient; a recommendation of performing one or more medical procedures on the patient; and a recommendation of performing one or more additional medical tests on the patient.

12. The computer-implemented method of claim 10, wherein the generated updated alert comprises an updated recommended treatment for the patient and the updated recommended treatment comprises at least one of: a recommendation of administering an effective dose of a pharmaceutical composition to the patient; a recommendation of increasing a dosage of an existing pharmaceutical composition being administered to the patient; a recommendation of performing one or more medical procedures on the patient; and a recommendation of performing one or more additional medical tests on the patient.

13. The computer-implemented method of claim 7, further comprising:
determining, based on the comparison with the stress profile, an urgency level for the patient corresponding to a level of stress for the patient;
wherein at least the first generated alert further comprises the urgency level determined for the patient.

14. The computer-implemented method of claim 8, further comprising:
reconfiguring the stress detection system to enable a second combination of physiological sensors, wherein the second combination of physiological sensors is a subset of the plurality of physiological sensors operatively coupled to the smart cable sub-assembly, the second combination of physiological sensors being different from the first combination of physiological sensors;
receiving, from the second combination of physiological sensors of the first smart cable assembly, a second set of measured physiological parameters for the patient;
comparing the second set of measured physiological parameters with the stress profile;
determining, based on the comparison with the stress profile, whether the second set of measured physiological parameters indicate a detection of stress associated with a treatable medical condition;
generating at least a second alert based on the determined detection of stress associated with the treatable medical condition; and
transmitting at least the second alert to a healthcare specialist associated with the patient.

15. The computer-implemented method of claim 14, wherein the generated second alert comprises a second recommended treatment for the patient, the second recommended treatment comprising at least one of: a recommendation of administering an effective dose of a pharmaceutical composition to the patient; a recommendation of increasing a dosage of an existing pharmaceutical composition being administered to the patient; a recommendation of performing one or more medical procedures on the patient; and a recommendation of performing one or more additional medical tests on the patient.
